(19) United States
(12) Patent Application Publication
(10) Pub. No.: US 2011/0184275 A1
(43) Pub. Date: Jul. 28, 2011

(54) Method for Operating a Computer System for Supporting an Insertion of a Guide Wire into Body Tissue and Associated Arrangement

(76) Inventor: Klaus Klingenbeck, Aufsess (DE)

(21) Appl. No.: 13/013,129

(22) Filed: Jan. 25, 2011

(30) Foreign Application Priority Data
Jan. 26, 2010 (DE) 10 2010 005 744.4

(51) Int. Cl. A61M 25/092; A61B 5/05

(52) U.S. Cl. 600/424; 604/95.01

(57) ABSTRACT

A method for operating a computer system for supporting an insertion of a guide wire into body tissue is proposed. An interventional procedure is prepared for by the insertion of the guide wire into body tissue. The guide wire is fitted with sensors. The latter's signals are received using the proposed method, so that a comparison of actual values with target values is possible and a future target direction of movement can be derived.

### CROSS REFERENCE TO RELATED APPLICATIONS

This application claims priority of German application No. 10 2010 005 744.4 filed Jan. 26, 2010, which is incorporated by reference herein in its entirety.

### FIELD OF THE INVENTION

The invention relates to a method for operating a computer system, wherein the computer system is used to support the insertion of a guide wire into body tissue. The invention also relates to an arrangement for preparing for an interventional procedure. It further relates to a method for determining the position and orientation of a tip of a guide wire in body tissue.

### BACKGROUND OF THE INVENTION

To prepare for an interventional procedure such as for example the insertion of a catheter, with the aid of which a dilatation balloon or a stent is passed into a body, a guide wire is regularly inserted into the body tissue. As its name suggests, the guide wire is used to guide the catheter, which is simply pushed over the guide wire. The guide wire is a thin, flexible wire. A guide wire is used to probe a target site in a body tissue of the patient.

It is known for such a guide wire to be moved in the body tissue using electromagnetic fields. WO 2006/005013 A2 describes how this “navigation” in the body tissue can be performed semi-automatically. To this end x-rays of the body to be transported are taken.

Taking these x-rays is complex.

### SUMMARY OF THE INVENTION

It is the object of the invention to demonstrate a way to support the insertion of a guide wire into body tissue in a non-complex manner.

The object is achieved in one aspect by a method for operating a computer system to support the insertion of a guide wire into body tissue, in another aspect by an arrangement for preparing for an interventional procedure, and in another aspect by a method for determining the position and orientation of a tip of a guide wire in body tissue, as claimed in the claims.

The inventive method for operating a computer system comprises the steps:
receipt of signals from a sensor device on the tip of the guide wire by the computer system, via which an actual location and an actual orientation of the tip of the guide wire can be transmitted,
comparison of the actual location and the actual orientation with a target location and a target orientation and determination of a target direction of movement for a future target movement of the guide wire.

By using one or more sensors on the tip of the guide wire, the signals of which can be and are received by the computer system, x-ray images can be dispensed with.

A target location and a target orientation can be determined in advance on the basis of inputs into the computer system or can be obtained automatically by the computer system itself on the basis of other information such as e.g. image information.

In a possible alternative the target direction of movement determined is communicated via an output device, so that a person giving the treatment, whose task is to insert the guide wire into the body tissue of the patient, immediately receives the details of what to do next, i.e. how the guide wire should be further inserted.

In another alternative the computer system generates control commands, as a function of the target direction of movement, and outputs these to means for movement of the guide wire. These can be means which at least include a magnetic coil, so that the magnetic field effects a movement of the tip of the guide wire.

In a preferred embodiment the inventive method includes the receipt of an input via a user interface to determine a size of a correction variable which is used when determining the target direction of movement. For example, the target direction of movement can have a component corresponding to the target orientation and a component perpendicular thereto, and a prefactor which determines the variable of the component perpendicular to the target orientation can be determined on the basis of an input. A user can in this way determine the slope of the correction of a deviation from the desired trajectory.

The inventive arrangement for preparing for an interventional procedure has a guide wire which includes at least one sensor for determining the position and orientation of the tip of the guide wire, and which is designed to emit sensor signals. It further has means for moving the guide wire in a tissue, and it has a control unit with means for receiving and evaluating the sensor signals and for determining and emitting control commands to the means for movement.

The arrangement thus reflects the concept in which the actual position and actual orientation of the tip of the guide wire are automatically sensed by at least one sensor on the guide wire itself, and in which in accordance with the inventive method a computer system evaluates these signals and in accordance with one aspect of the inventive method immediately effects the correction movement.

The means for movement preferably here include at least one magnetic coil.

The inventive method for determining the position and orientation of a guide wire in body tissue includes the acquisition of measuring signals by at least one sensor arranged on the guide wire, the sending of measuring signals by the sensor, the receipt of the measuring signals by a computer unit and the subsequent evaluation of said measuring signals by the computer unit.

### BRIEF DESCRIPTION OF THE DRAWINGS

A preferred embodiment of the invention is described below, with reference to the drawing, in which

FIG. 1 schematically shows the components of an inventive arrangement for preparing for an interventional procedure,

FIG. 2 shows a flow diagram of two alternative embodiments of the inventive method and

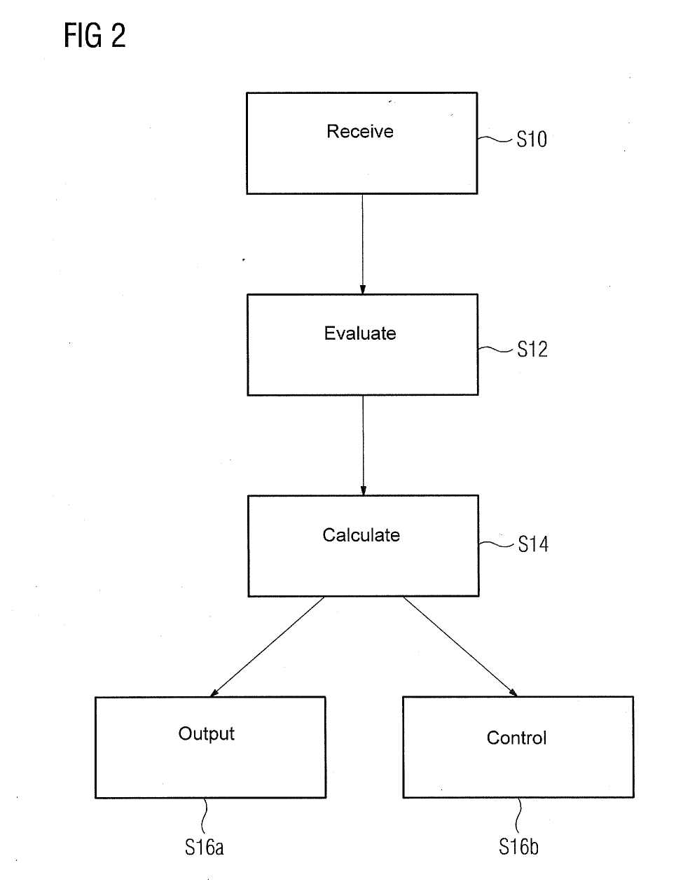

FIG. 3 is a graphical representation explaining the variables used in the description of the inventive method.

### DETAILED DESCRIPTION OF THE INVENTION

An arrangement designated overall by 100 for preparing for an interventional procedure includes a guide wire 10, on which a plurality of coils is arranged, of which two coils 12a, 12b are shown by way of example. With the aid of coils a position and an orientation of the front region 14 can be measured.

One coil is normally provided for this for each component of the three-dimensional space.

The guide wire 10 should be inserted into the body tissue of a patient (not shown) and should be automatically movable there. To move the guide wire, magnetic fields are generated, as a result of which the guide wire 10 consisting of ferromagnetic material is attracted or repelled. The magnetic fields are generated by a coil shown schematically by 16, which is supplied with current by a current source 18. The size of the current is determined by a computer unit 20. The measured values of the sensor coils 12a, 12b are fed to the computer unit 20 via cables 24 arranged in a cavity 22 of the guide wire 10. Inputs can be made at the computer device 20 using a computer mouse 26 or a keyboard 28. Images 32 can be displayed on a screen 30.

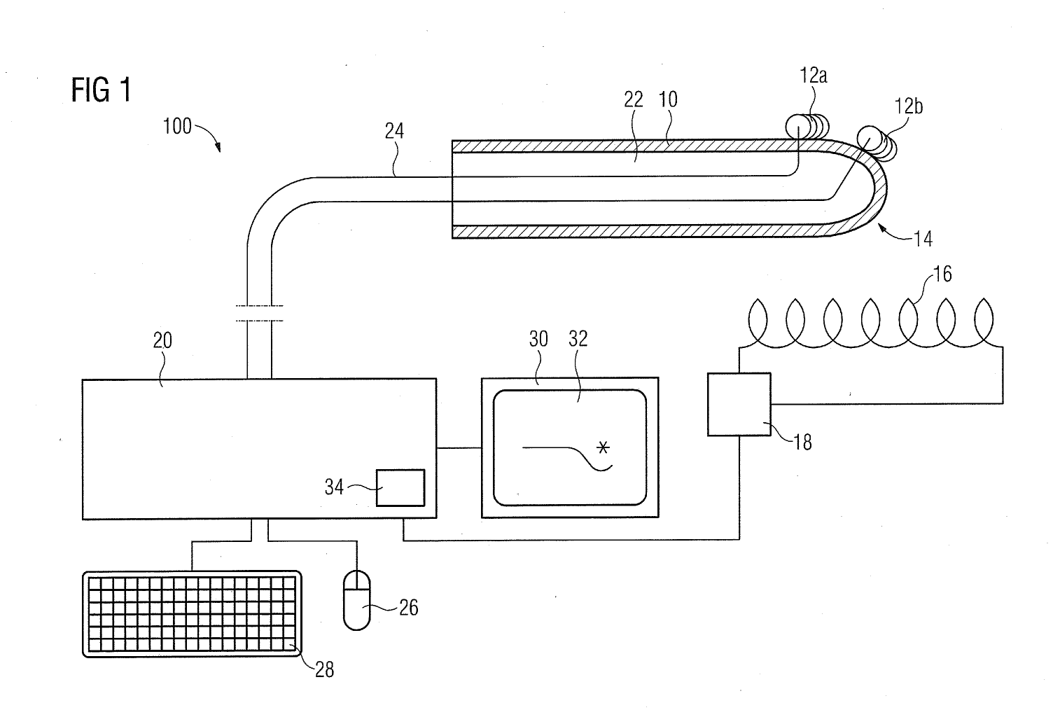

It will now be shown how the inventive method is executed, said method being performed by the computer unit 20 and the technical devices linked thereto. A receiver unit of the computer unit 20 (not shown in FIG. 1) receives the signals from the sensor coils 12a, 12b in accordance with step S10. The computer unit 20 then evaluates the incoming signals with the aid of a microprocessor 34 in accordance with step S12 and then in step S14 calculates the current to be applied to the coil 16. In a first alternative a display is output on the screen 32 showing how the guide wire 10 should be further moved, in accordance with step S16a. In a second alternative a signal is automatically emitted by the current source 20 to the current source 18 indicating the current to be supplied to the coil 16, in accordance with step S16b.

In the computer unit 20 a navigation path NP is in particular laid down. This can be determined by a user for example on the basis of x-ray images of the patient taken previously; for example, the user can mark particular places in an x-ray image and in this way determine the path. Alternatively the central line of a body vessel can be automatically determined by the computer unit 20 on the basis of medical images such as x-ray images, nuclear resonance images, etc. Based on a representation of vessels, the path of least resistance through tissue can additionally be automatically determined.

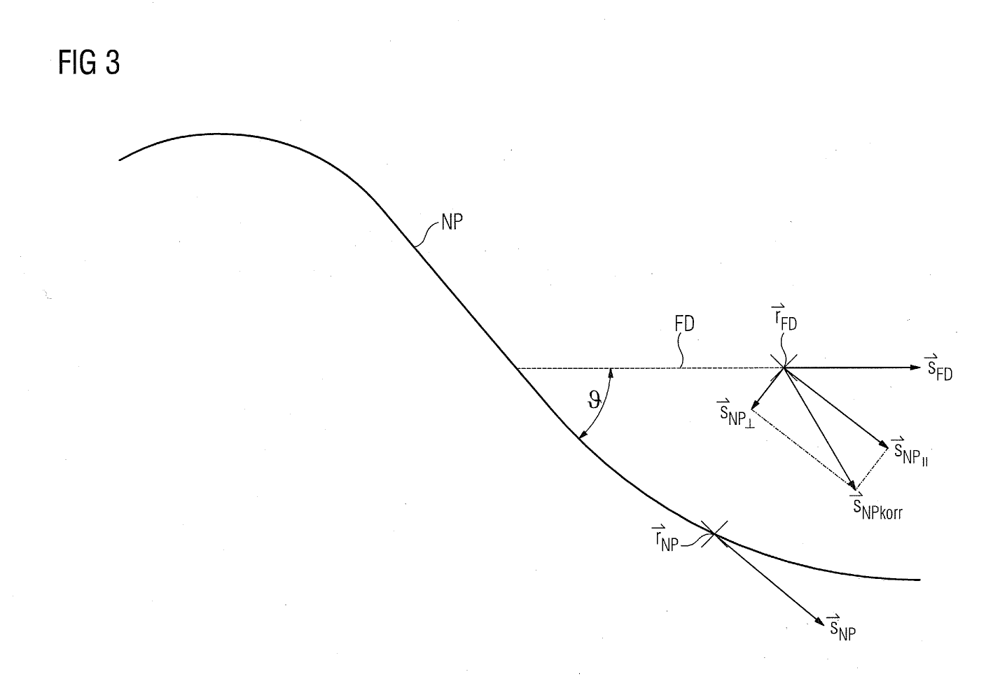

In accordance with FIG. 3, now let the guide wire be moved on a curve FD which deviates from the navigation curve NP in one region. Let an actual location r_{FD} deviate from the target location r_{NP}. In the same manner, also let actual orientation S_{FD} deviate from a target orientation S_{NP}. The guide wire should now subsequently be moved in accordance with the target orientation S_{NP} on the one hand, and on the other hand the deviation between actual location and target location should be equalized. In this way a target direction of movement S_{NPkorr} can be assembled from a component S_{NP} and a component perpendicular to the target orientation S_{NP}, S_{NP}_{_{⊥}}.

The length of S_{NP} can here be equal to the length of S_{NP}, whereas the length of S_{NP}_{_{⊥}} can be proportional to the angle θ which occurs in a triangle, the sides of which pass through the points r_{Np} or r_{FD} in the directions S_{NP} or S_{FD}. Alternatively the perpendicular component S_{NP}_{_{⊥}} can also be proportional to the sine of this angle θ, sin θ. A possible proportional factor can if necessary be input via the user interfaces 26, 28 by an operator.

The computer unit 20 now calculates the variable S_{NPkorr}.

The role of the magnetic coil 16 is to generate such a magnetic field that is parallel to the direction arrow S_{Npkorr}, so that the guide wire 10 can be moved in this direction and hence approximates to the normal path NP.

The inventive method performed on the computer unit 20 hence enables automatic navigation of the guide wire 10 in the body tissue of a patient. To this end the computer unit 20 must interact with the guide wire 10 and the means 16, 18 for movement of said guide wire, and must receive and/or output signals.

The method for operating a computer system, as described above, essentially functions even if sensor signals from the computer unit are merely simulated and the receipt of control signals is likewise merely simulated. The computer unit 20 can hence also essentially be operated without the guide wire 10 and the means 16, 18 for movement of said guide wire, although in that case there is of course no obvious perceived effect on a guide wire 10 in the patient tissue.

Instead of using magnetic fields, an electromechanical guidance system in accordance with the Hansen principle can also be used.

1.-7. (canceled)

8. A method for operating a computer system for supporting an insertion of a guide wire into body tissue, comprising:
receiving signals from a sensor device at a tip of the guide wire by the computer system;
determining an actual location and an actual orientation of the tip of the guide wire based on the signals;
comparing the actual location and the actual orientation with a target location and a target orientation; and
determining a target direction of movement the of the guide wire based on the comparison.

9. The method as claimed in claim 8, wherein the target direction of movement is communicated via an output device.

10. The method as claimed in claim 8, wherein control commands are emitted for moving the guide wire based on the target direction of movement.

11. The method as claimed in claim 8, wherein an input is received via a user interface to determine a size of a correction variable used in determining the target direction of movement.

12. An arrangement for preparing an interventional procedure, comprising:
a sensor device that emits signals;
a guide wire comprising the sensor device at a tip of the guide wire;
a device that moves the guide wire in a body tissue; and
a computer system that:
receives the signals,
determines an actual location and an actual orientation of the tip of the guide wire based on the signals,
compares the actual location and the actual orientation with a target location and a target orientation,
determines a target direction of movement the of the guide wire based on the comparison, and
emits control commands to the device for moving the guide wire in the body tissue based on the target direction of movement.

13. The arrangement as claimed in claim 12, wherein the means for movement include at least one magnetic coil.

14. A method for determining a position and an orientation of a tip of a guide wire in body tissue, comprising:
arranging a sensor device on the tip of the guide wire;
emitting signals by the sensor device;
receiving the signals by a computer system;
determining an actual location and an actual orientation of the tip of the guide wire based on the signals;
comparing the actual location and the actual orientation with a target location and a target orientation; and
determining a target direction of movement the of the guide wire based on the comparison.
